(19) United States
(12) Patent Application Publication
(10) Pub. No.: US 2018/0166169 A1
(43) Pub. Date: Jun. 14, 2018

(54) METHOD, APPARATUS AND SYSTEM FOR REGISTERING, PHOTOGRAPHING, TRACKING AND MANAGING SURGICAL INSTRUMENTS AND EQUIPMENT

(71) Applicants: Daniel J. Mastromonaco, Traverse City, MI (US); Antonio G. Mastromonaco, Plymouth, MI (US)

(72) Inventors: Daniel J. Mastromonaco, Traverse City, MI (US); Antonio G. Mastromonaco, Plymouth, MI (US)

(21) Appl. No.: 15/891,964

(22) Filed: Feb. 8, 2018

Related U.S. Application Data
(63) Continuation of application No. 15/157,913, filed on May 18, 2016, now abandoned.
(60) Provisional application No. 62/164,713, filed on May 21, 2015.
(60) Provisional application No. 62/143,071, filed on May 19, 2015.

(51) Int. Cl. G16H 40/40; G06Q 50/22

(52) U.S. Cl. CPC G16H 40/40; G06Q 50/22

(57) ABSTRACT

A method, apparatus and system for registering, photographing, tracking and managing surgical instruments and equipment by multiple users. An integrated check-in & check-out station is provided to check medical equipment into a health care facility, take photos, provide labels, and create a cloud based record of the equipment delivered, including photo(s) of equipment checked in. The record created is accessible by the medical equipment vendor, or any linked vendor, and by medical facility staff users, from any location. The contents of any equipment tray can be identified simply by scanning the code on the label printed at the check-in station and applied to the equipment tray.

### REFERENCE TO RELATED APPLICATIONS

This application claims priority to U.S. provisional patent application Ser. No. 62/164,713 filed on May 21, 2015, and Ser. No. 62/143,071 filed on May 19, 2015, both under the same title as the present application.

### FIELD OF THE INVENTION

The present invention relates to the field of managing loaned medical instrumentation and equipment.

### BACKGROUND OF THE INVENTION

Loaned instrumentation or medical equipment are typically received and handled by sterile processing department staff in conjunction with healthcare facility staff (hereafter referred to as “Staff”). The Staff is responsible for logistics, handling, communication, cleaning, sterilization and storage of surgical instrumentation, medical equipment, or other physical inventory (hereafter referred to as “Equipment”) that is borrowed, leased, or consigned from third party vendors, couriers (hereafter referred to as “Vendors”). Instrumentation and equipment not owned by the hospital, surgery center, or other healthcare facility (hereafter referred to as the “Facility”) and received from Vendors is considered contaminated upon arrival. The Staff is responsible for returning the Equipment to a sterile state. Due to the unpredictable travel patterns of this Equipment entering and leaving different facilities, the arrangement and presence of the contents of the Equipment are inconsistent.

Historically, this Equipment has been documented manually using written log books, identification tags, or other non-electronic means. Once manually documented, facilities lack an accurate representation or easily accessible electronic records with details for the Equipment. In an effort to improve accuracy, some facilities have taken it upon themselves to manually take photographs of the Equipment using digital or film cameras. Matching photographs, if any, with manually documented information has proven to be an inefficient process for the Facility. Conventional processes are very time-consuming for Staff and Vendors and are prone to error.

### SUMMARY OF THE INVENTION

The present invention comprises methods and apparatus wherein a user uses a check-in & check-out station to check medical equipment into a health care facility, take photos, provide labels, and create a cloud based record of the equipment delivered, including photo(s) of equipment checked in. The record created is accessible by the medical equipment vendor, or any linked vendor, and by medical facility staff users, from any location.

These and other aspects of the invention will be more fully understood and appreciated by reference to the Description of the Preferred Embodiments, and appended drawings.

### BRIEF DESCRIPTION OF THE DRAWINGS

FIG. 1A illustrates a front view of one example of a check-in & check-out station.

FIG. 1B illustrates a side-corner view of one example of a check-in & check-out station.

FIG. 2 illustrates an exemplary flow chart for processes related to the operation of the check-in & check-out station software.

FIG. 3 illustrates an exemplary flow chart for processes related to the operation of the remote access administration panel software.

FIG. 4 illustrates an example of a label or tag printed upon check-in.

FIG. 5 illustrates an example of a printable PDF report generated at the request of a user. This report may be accessed through active links while viewing Equipment and active links sent through email or SMS text message communications.

FIG. 6 illustrates an example of two-way SMS text message communication on a mobile device.

FIG. 7 shows the various software modules of a preferred embodiment computer software.

### DETAILED DESCRIPTION OF THE PREFERRED EMBODIMENTS

### Overview

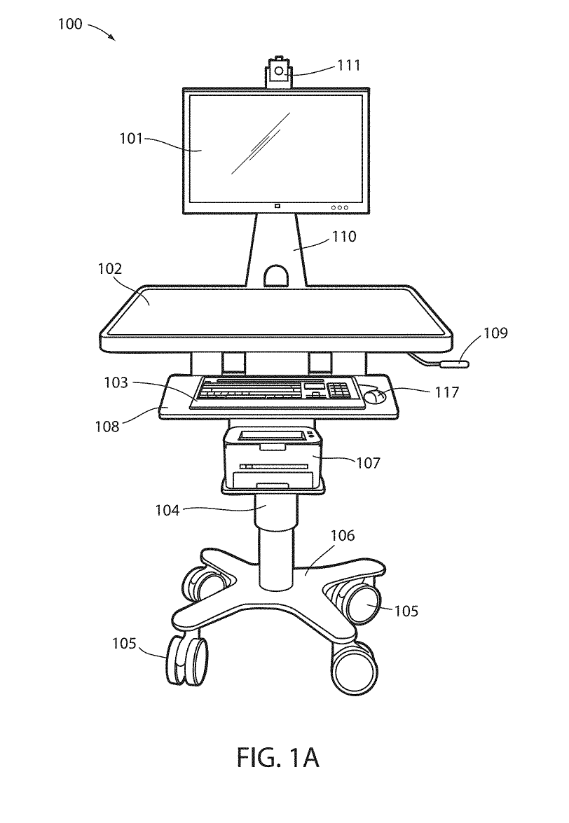

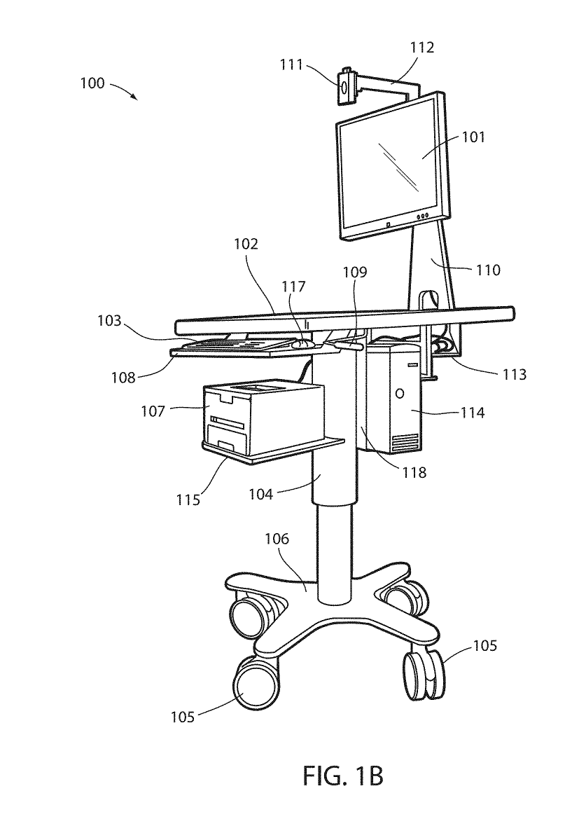

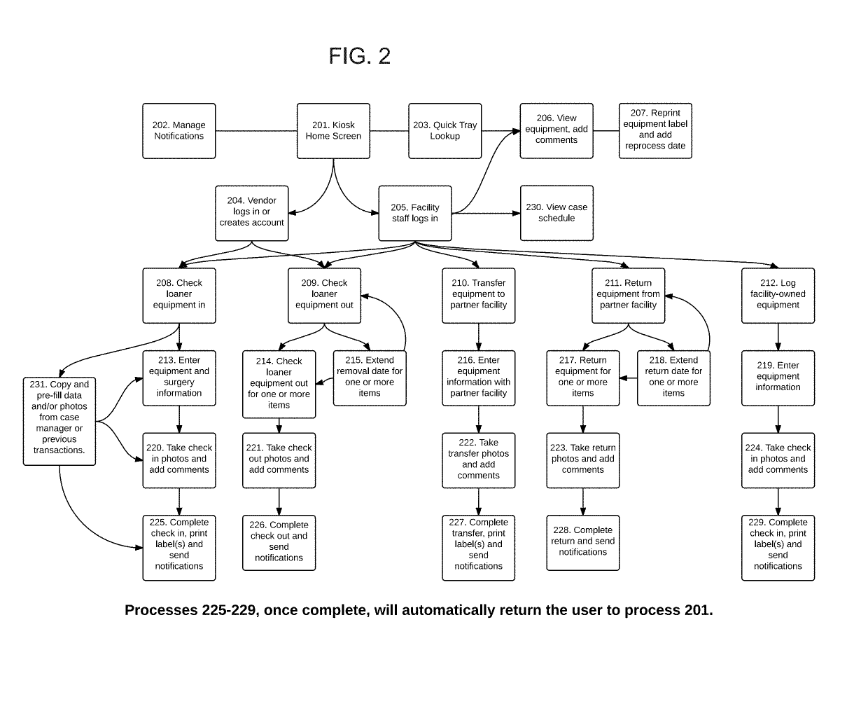

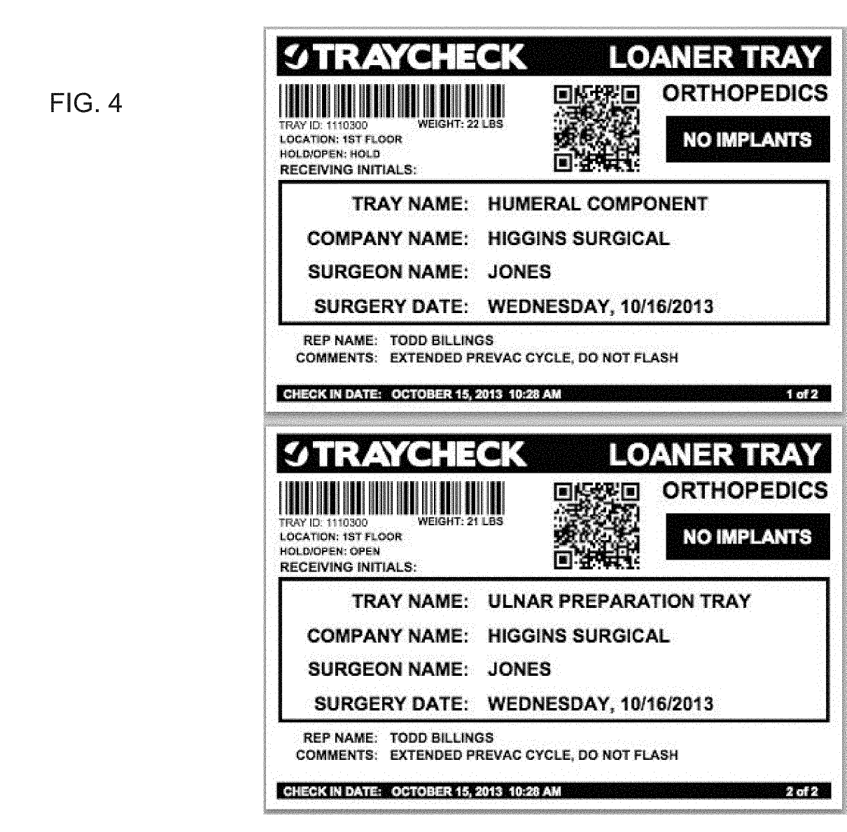

In the preferred embodiments, a check-in & check out station 100 (sometimes referred to as a “kiosk”) is used by a vendor to check medical equipment into a health care facility using computer 114, take photos via camera 111, provide labels (FIG. 4) via printer 107, and create a cloud based record of the equipment delivered, including photo(s) of equipment checked in (FIGS. 1 and 2). Options for requiring additional information are also provided. FIG. 2 shows the process steps performed at kiosk 100.

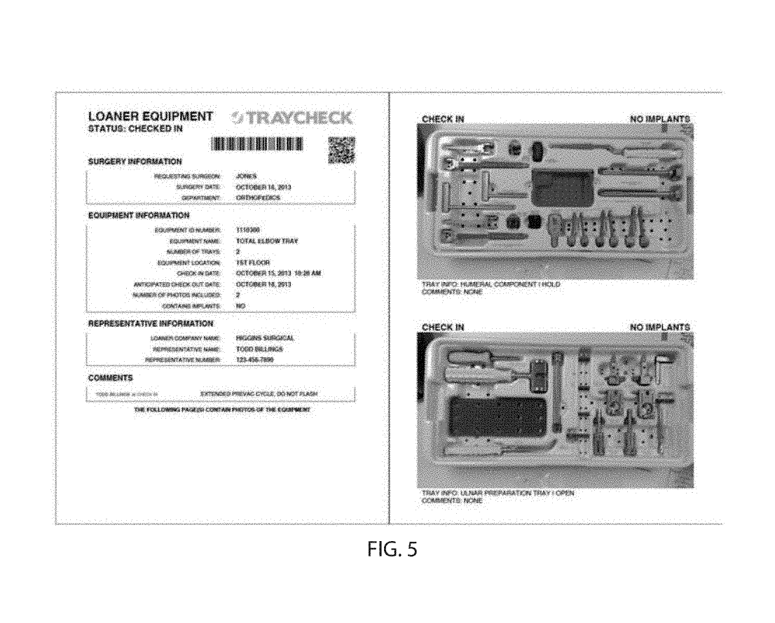

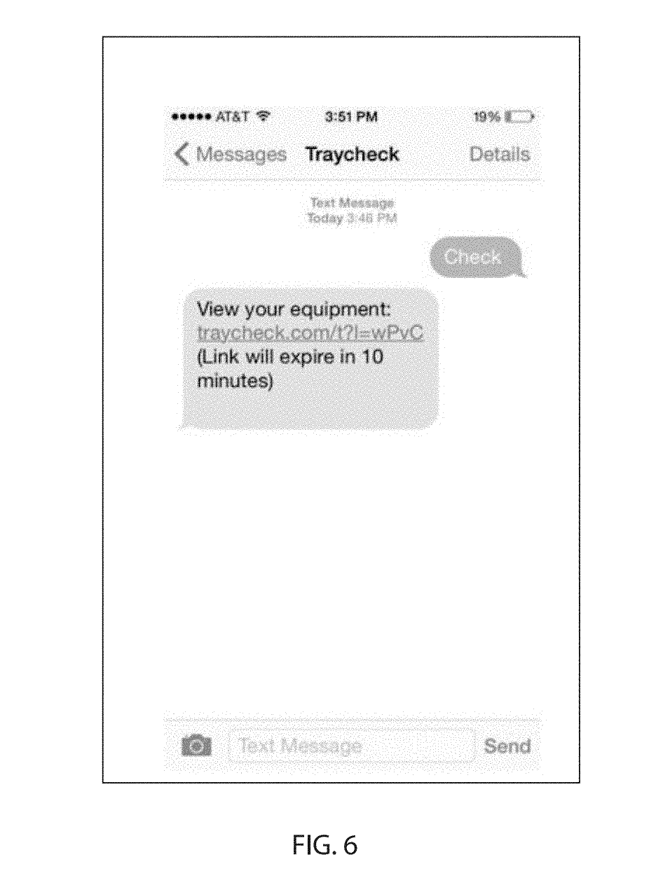

The record created at check-in is accessible for use by the medical equipment vendor, or any linked vendor, and by medical facility staff users, from any location, via the Admin Panel 301. Reports (ex. FIG. 5) can be displayed or printed, and various notifications (FIG. 6) are sent to appropriate users of the system.

### The Check-in & Check-Out Kiosk

FIGS. 1A-1B illustrate a check-in & check-out kiosk workstation apparatus 100 of a preferred embodiment. The system is comprised of a work surface 102 mounted on a gas-powered cylindrical adjustable upright 104 and resting on a steel base 106 with rubber caster wheels 105. The work surface 102 is of adequate size to accommodate Equipment of various shapes and sizes. The height of the apparatus can be adjusted using a release lever 109 mounted underneath the work surface 102. The gas-powered cylindrical upright 104 acts as a mounting platform for a printer bracket 115 and a computer bracket 116. Additionally, the work surface 102 acts as a mounting platform for a slide-out keyboard and mouse tray 108, a cord storage bracket 113, and an upright screen mounting bracket 110. Furthermore, the upright screen mounting bracket 110 acts as a mounting platform for an overhead camera mounting bracket 112. One example of a suitable workstation is a LUND DataCart™ DCT-1RLT with a standard work surface 32″ width by 24″ deep by 1.25″ high. It is also possible to build a custom workstation to house the electronic components of the system. Other types of carts, workstations, desks, wall-mounted units, or other apparatuses may be employed in some embodiments of this system.

The computer bracket 116 is equipped with a computer 114 suitable for controlling operations within the system. The overhead camera mounting bracket 112 is equipped with a USB-powered high-definition camera 111 connected to the computer 114. The camera 111 and camera bracket 112 are oriented in a fixed position in order to properly capture overhead photographs of the Equipment. Examples of a camera suitable for this system include a Logitech™ C920, a Microsoft™ Lifecam Studio, or similar cameras allowing high resolution image capture. The upright screen mounting bracket 110 is equipped with a monitor 101 suitable for viewing content from the computer 114. The monitor 101 displays content from the computer 114 in an adequate format to allow the user to interface with the system. The slide-out keyboard and mouse tray 108 houses a standard computer keyboard 103 and computer mouse or touch pad 117 interfacing with the computer 114. The keyboard 103 and mouse 117 provide data to the computer 114 producing detailed descriptions of Equipment presented. The printer 107 rests on the printer bracket 115 and interfaces with the computer 114. Examples of a suitable printer for this system include the Zebra™ GC420T or other compatible thermal-transfer ribbon-based label or tag printers. Additionally, the system may incorporate a barcode scanner, QR code scanner, RFID tag identifier, or other near-field communication device to assist in identifying, tracking, or managing the Equipment. Thus for example, a user can scan the barcode or QR code on a wrapped sterilized tray, and thereby obtain a readout identifying the contents of the tray, without having to open it and then re-sterilize. This can be done at the kiosk or through the admin portal from any location. It enables Staff and vendors to confirm that the tray contains everything necessary for a forthcoming surgery. Furthermore, the system may incorporate other components that may assist in Equipment data collection, such as a scale, radiographic or other imaging device.

### Check-in & Check-Out Station Operations

Medical equipment trays are checked in and checked out by vendors at check in & check out kiosk 100. At check in, the vendor or someone acting on his or her behalf, enters information prompted by a check in screen, concerning the tray(s) being checked in. Check in and check out dates are entered. The trays are photographed and labels are printed, which may accompany or be affixed to the trays. The system provides notifications to various users, including a reminder notification when a tray is scheduled for check out. At check out, check out information is entered and additional photographs of the tray may be taken. The kiosk vendor functions can also be used by facility staff for facility generated transfers of trays to and from one facility location to another.

Check-in functions, and the similar transfer of equipment from one facility to another, may be performed through the kiosk. Some operations which may be performed from anywhere can also be performed at the kiosk. FIG. 2 is an exemplary flow chart illustrating the software utilized in the kiosk interface under a preferred embodiment. In this embodiment, the computer 104 is configured in such a way that allows the user to only view the contents of a unique web page housed on a server containing pertinent information defined by each Facility. Components of a traditional web browser, including, but not limited to, status bars, menu bars and resizable frames have been hidden from the user (“kiosk mode”). These components have been removed to streamline the system and prevent the user from accessing other web pages or other components of the computer 104. Additionally, the system is designed to automatically return to the home screen 201 after events of power failure, loss of connectivity, network outage or other disruptions are resolved. In another embodiment, the user may access the server through additional devices capable of network access, including, but not limited to, laptop computers, tablets, or other application-based mobile devices. These and other suitable devices may act as or be used in lieu of a check-in and check-out station while still utilizing the same software.

In the preferred embodiments, the home screen 201 allows a user to log in as a Vendor 204 or Staff 205. Additionally, previously unregistered Vendors may create an account 204. Beginning at the home screen 201, users can also manage communication and notification settings 202 or look up equipment details and photos 203 by clicking links at the top corners of the page. Under the manage notifications link 202, users have the opportunity to select communication preferences from email to SMS text messaging based on specific Facility departments. The equipment lookup feature 203 allows users to view equipment 206, add comments if logged in 206, and reprint labels 207. The system also provides an “auto-fill” capability 231, whereby information pertinent to a particular tray which has been previously entered into the system auto-fills that information for the vendor as the tray is checked in. Tray and case information may be entered into the case manager 315 or automatically saved from previous check in transactions. Staff may view the case schedule once logged in 230.

After a Vendor logs in or creates an account, or Staff logs in and chooses to check equipment in 208, the user is led through a sequence to complete the check-in process. The system will detect if a Vendor has Equipment checked in that has lapsed its anticipated checkout date, and if so, will prompt the Vendor to follow the checkout process first. If no such onsite Equipment exists in the system, the Vendor will be allowed to continue the check-in process. Equipment information is entered 213 beginning with the number of trays the user is checking in. The system allows a single tray or multiple trays to be checked in under a single check-in process. The system will recognize the name and quantity entered for the equipment and search the database to make recommendations to the user. These recommendations allow the user to re-use previously entered information 231 to prefill data fields and save time. Alternatively, users may copy Equipment and case information from previously entered information 231 in the case manager 315, which is accessible to both Vendors and Staff in the admin panel 301. Equipment information also may include, but is not limited to, surgeon name, field of practice, surgery date, surgery time, procedure performed, operative side, patient initials, user representative name, user company name, loaner fee, tray weight, tray dimensions, directions for use, instructions for processing, instructions for handling, list of contents, consignment status, or other pertinent information as deemed necessary by the Facility or Vendor to properly track the Equipment.

At this point, the system activates the camera 111 automatically and allows the user to take unlimited photographs of the Equipment 220. Equipment is placed on the work surface 102 for image capture. Additional details or individual tray names are entered if desired or required by the Facility. The system additionally allows the user to reframe the image to be captured with a digital zoom feature. Photos are uploaded to a web server to be stored for future access.

To complete the check-in process, the user enters an anticipated checkout date and additional information as desired or required by the Facility 225. Such information may include, but is not limited to, purchase order number, tray location at the Facility, count sheets, sterilization instructions, additional users to be notified, additional comments on the Equipment, assignment of Equipment to another user, or other pertinent information as the Facility deems necessary to track the Equipment. Upon completion, the system uploads collected data to the server, automatically prints a label or tag FIG. 4 for each tray and sends communications according to user notification preferences. Labels include all necessary information as deemed appropriate by each Facility. The system also allows for labels to print remotely to networked printers.

Similar to the check-in process, Staff may use the same sequence to transfer Equipment to a partnering Facility 210, 216, 222, 227. Staff designates which Facility the Equipment should be transferred to 216 and additional notifications are sent to Staff at the partnering Facility 227.

After a Vendor or Staff logs in and chooses to check Equipment out 209, the system will search for any Equipment currently on site. For Vendors, the system will search for Equipment assigned to that Vendor or any other user the Vendor is linked to. For Staff, the system will search for all Equipment on site for the Facility. If no such onsite Equipment is found, the user will not be able to continue. If onsite Equipment is found, the Equipment is listed and the user may select one or multiple items to check out now 214 or extend the removal date and leave on site for a pre-determined period of time 215. The system will update the anticipated checkout date of any items selected for extension and either return the user to the beginning of the process 208, 209 or continue the checkout process with the items the user has selected to check out.

After continuing the checkout process, the system will automatically activate the camera 111 and allow the user to take unlimited photographs of the Equipment for comparison as deemed necessary by the Facility 221. The user may enter any additional comments before completing the checkout process 226. The system will upload collected data to the server and send communications based upon user notification preferences.

Similar to the checkout process, Staff may use the same sequence to return Equipment from a partnering Facility 211, 217, 218, 223, 228. Notifications are sent to Staff at the partnering Facility 227 upon return.

The Facility may, at its discretion, activate an additional feature of the system that allows them to document Equipment owned by the Facility 212 using the same sequence as the check-in process 208. The exception to this sequence is that Equipment information may not necessarily contain specific surgery information or information used for loaner instrumentation 219.

### Admin Portal Operations

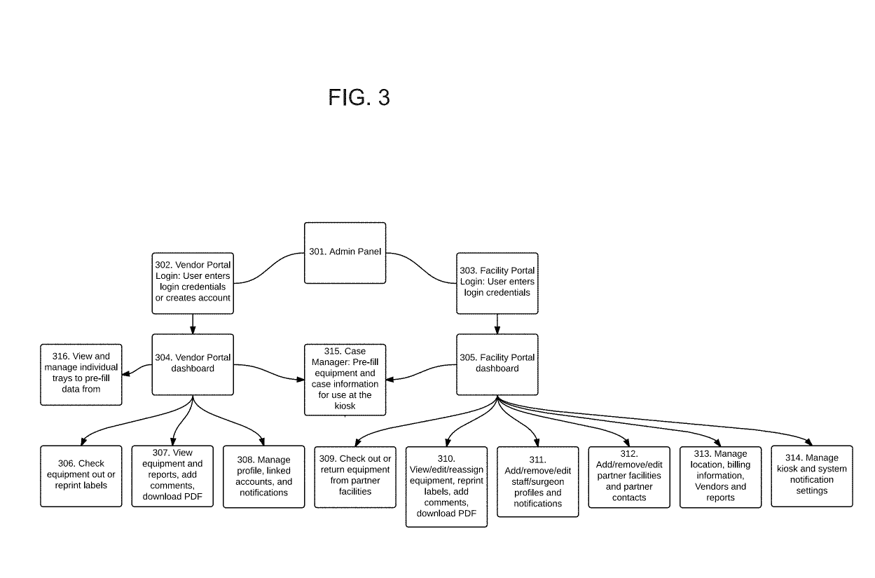

There are numerous operations which can be conducted by staff or vendor users from any location, including the kiosk. FIG. 3 is an exemplary flow chart illustrating the software utilized in the administration panel 301 under a preferred embodiment. Vendors and Staff utilize the administration panel 301 to access data collected by the kiosk software FIG. 2. Vendors and Staff may also update data, print documents, and send communications via the administration panel 301.

Vendors may log in using a web-enabled device by entering a username and password or create an account 302. Upon logging in, Vendors are sent to a filterable dashboard 304 where they may check Equipment out 306, view Equipment and reports, add comments, and download a dynamically generated PDF FIG. 5. Additionally, Vendors may manage profile settings, linked accounts, and notification preferences 308. Vendors may check Equipment out 306 only if that Equipment is on site and if the Facility permissions allow. Vendors may view, comment on, or download a PDF report for the Equipment assigned to them and to linked user accounts 307. Vendors may generate reports identifying all equipment which they have at a particular facility. Vendors may also pre-load and edit individual tray information to reference for future transactions within the system 316.

Upon completion of Equipment checkout or other modifications of data 306-308, communications may be sent via email, SMS text message, or other means to users based on notification preferences. The system also allows for two-way communication FIG. 6 to complete functions such as checkout using SMS text messaging.

Staff may access the administration panel 303 by entering login credentials using any web-enabled device. Similar to the Vendor portal 304, Staff may view a filterable dashboard 305 with multiple options to view or manipulate data. This dashboard contains data that can be actively sorted based on multiple fields. Equipment may be checked out or returned from partnering Facilities 309 by selecting the Equipment from the dashboard. Staff may also view, edit or reassign Equipment details, add comments, reprint labels, and download dynamically generated PDF reports 310. Staff may manipulate data for other Staff members and surgeons, including, but not limited to, adding, removing, editing, or updating notification settings 311. Staff may request the addition or removal of partner Facilities 312 to facilitate the transfer and return of Equipment to and from affiliated Facilities. The system allows additional contacts to be entered for each partnering Facility to receive communications taking place in the transfer process FIG. 2.

Staff may manage Facility and billing information, Vendors and reports 313. Facility location and billing information includes, but is not limited to, physical address, shipping address, billing address, and subsequent personnel for contact. Staff may view Vendor data in aggregate or individually, manage Vendor permissions, and request specific Equipment from Vendors. Staff may also run unlimited printable reports by defining unique parameters.

Staff may manage settings 314 that define the system processes utilized by Vendors or Staff while using the kiosk software FIG. 2. These settings may include, but are not limited to, hiding or showing specific features or data, requiring additional information to be entered, and modifying the way processes function. For example, the Facility may require a purchase order number be entered, subsequently adding to the data that is uploaded to the server, displayed to the user, and communicated through electronic or printable content. The kiosk setting options allow for hiding, showing or requiring data fields that may include, but are not limited to, surgery date, surgery time, patient initials, loaner fee, tray weight, tray location, tray availability, purchase order number, instrument list, and sterilization instructions. Staff may also customize options including, but not limited to, Equipment lookup, photograph parameters, and time policies. Additionally, Staff may manage notification settings based on time parameters defined by the Facility.

Staff and Vendors may also pre-fill Equipment and surgery case information using the case manager 315 from any web-enabled device. The information entered in the case manager will allow users to quickly select the Equipment to be processed without the need for manual reentry 231. Pre-filled information from the case manager may also be used for extra communications between the Facility and Vendor, such as to track which expected Vendors have already visited and which have yet to arrive for a particular time period.

Upon completion of manipulation of data 309-313, communications may be sent via email, SMS text message, or other means to users based on notification preferences. The system also allows for two-way communication FIG. 6 to complete functions such as checkout using SMS text messaging.

Integration with Other Systems

The system also includes an integration function. This allows integration with other management and tracking software already in use by the facility or other third party applications that may complement the system.

### Software Architecture

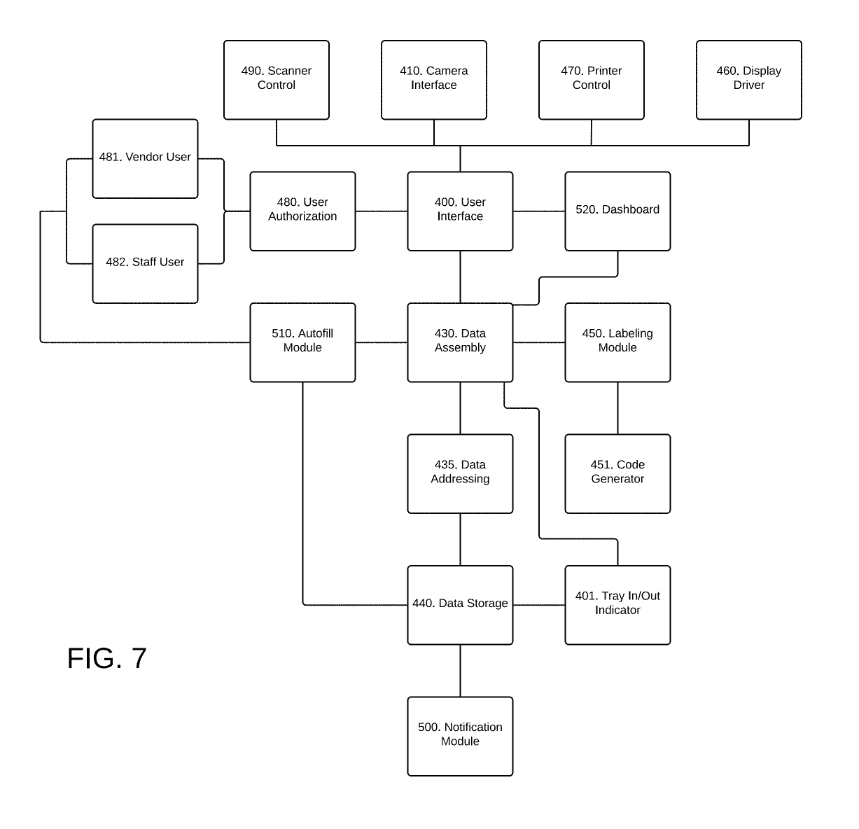

FIG. 7 illustrates the computer software architecture used for tracking medical equipment into and out of a health care facility. A user interface module 400 facilitates use of software by a vendor or staff user checking medical equipment into and out of a health care facility or moving equipment within the facility. User information is stored on a vendor user table 481 or a staff user table 482, and users are authorized via a user authorization module 480. User interface module prompts the user for entry of additional data information selected from the group consisting of the surgeon name, field of practice, surgery date, surgery time, procedure performed, operative side, patient initials, user representative name, user company name, loaner fee, tray weight, tray dimensions, directions for use, instructions for processing, instructions for handling, list of contents, consignment status, or other pertinent information as deemed necessary to properly track the Equipment.

Display driver 460 facilitates display of user interface information. A camera linking module 410 is provided for linking the software to a camera used for taking at least one photo of medical equipment being checked in. Data assembly table 430 facilitates assembly of data entered via user interface module 400, including photographs taken of said medical equipment and for receiving data entered identifying said medical equipment being checked into or out of a health care facility. It also assembles data retrieved from data storage module 440 through user interface 400. Data addressing module 435 addresses data and transfers it into data storage module 440 and facilitates retrieval of the data for subsequent use. A tray in or tray out indicator module 401 is linked to data storage module 440 and assembly module 430 to indicate whether equipment identified by data in data storage module 440 is “in” or is “out.”

A labeling engine 450 is linked to and activated by said data assembly module 430, for preparing identifying labels for said medical equipment based on said data in said data assembly module. A code generator is linked to said labeling module whereby every label generated has a computer readable code printed on labels applied to the equipment. A printer control module 470 is provided to facilitate printing said labels for attachment to said medical equipment or a package containing said medical equipment. Scanner module 490 facilitates scanning the labeled equipment to identify it and facilitate confirmation of the appropriate contents.

An “auto-fill” module 510 is linked from data storage 440, staff and user vendor tables 481 and 482, and data assembly table 430, whereby information pertinent to particular equipment or surgical cases which has been previously entered into the system auto-fills that information for the user as equipment is checked in or checked out.

A dashboard display module 520 provides a filterable dashboard display with multiple options to view or manipulate data. The user interface module 400 is programmed to receive entry of anticipated check out dates for equipment, and to automatically notify the relevant users through a notification module 500.

### CONCLUSION

Thus, the present invention in its preferred embodiments provides a method, apparatus and system for registering, photographing, tracking and managing loaned or owned medical equipment by multiple users over a network or such as the Internet. An integrated check-in & check-out station allows a vendor to check medical equipment into the facility, take photos, provide labels, and create a cloud based record of the equipment delivered, including photo(s) of equipment checked in. This provides accuracy, and relieves the facility staff from having to enter and somehow track loaned equipment entering and leaving the facility. The record created is accessible by the medical equipment vendor, or any linked vendor, and by medical facility staff users, from any location, including the integrated check in & check out station. A simple scan of a code on the label applied to the equipment tray after sterilization enables a user to identify and/or confirm the contents of the wrapped tray.

One skilled in the art will appreciate that the various components of this system can be rearranged, omitted, or adapted in various ways depending on the requirements and preferences of each Facility, Vendor or other user of the system.

1. A system for tracking medical equipment into and out of a health care facility comprising:
A check-in & check-out station for medical equipment comprising:
a user interface,
a camera for taking at least one photo of the equipment,
a computer programmed for entering data concerning the equipment into the computer, accepting said photos of the equipment and linking said photos to said data, and preparing identifying labels for said equipment based on said data,
a viewable display,
and a printer operably connected to said computer to facilitate printing said labels for attachment to said equipment or a package containing said equipment;
a server for storing said data and said linked photos, said server being linked to said check-in & check-out station computer;
said server and said computer being programmed to provide authorized medical equipment vendors and medical facility users access to said data and photos from any internet enabled location and from said check-in & check-out station.

2. The system of claim 1 in which said computer is programmed to generate a computer recognizable code which is included with said data, and also to print said code on said label, whereby said code can be scanned by an authorized user or vendor to determine the contents of a package containing said equipment.

3. The system of claim 2 in which said check-in and check-out station includes a scanner for scanning and recognizing said code.

4. The system of claim 3 in which said computer is programmed to provide and allow users to select the option of receiving notifications of particular events related to equipment which is entered into the system.

5. The system of claim 4 which also provides an “auto-fill” capability, whereby information pertinent to particular equipment or surgical cases which has been previously entered into the system auto-fills that information for the user as equipment is checked in.

6. The system of claim 5 in which said computer is programmed to prompt for entry of additional data information selected from the group consisting of the surgeon name, field of practice, surgery date, surgery time, procedure performed, operative side, patient initials, user representative name, user company name, loaner fee, tray weight, tray dimensions, directions for use, instructions for processing, instructions for handling, list of contents, consignment status, or other pertinent information as deemed necessary to properly track the Equipment.

7. The system of claim 6 in which said computer is programmed to display filterable dashboards with multiple options to view or manipulate data.

8. The system of claim 7 which is programmed to receive entry of anticipated check out dates for equipment, and to automatically notify the relevant users.

9. The system of claim 1 which is programmed to receive entry of anticipated check out dates for equipment, and to automatically notify the relevant users.

10. The system of claim 1 in which said computer is programmed to provide and allow users to select the option of receiving notifications of particular events related to equipment which is entered into the system.

11. The system of claim 1 which also provides an “auto-fill” capability, whereby information pertinent to particular equipment or surgical cases which has been previously entered into the system auto-fills that information for the user as equipment is checked in.

12. The system of claim 1 in which said computer is programmed to prompt for entry of additional data information selected from the group consisting of the surgeon name, field of practice, surgery date, surgery time, procedure performed, operative side, patient initials, user representative name, user company name, loaner fee, tray weight, tray dimensions, directions for use, instructions for processing, instructions for handling, list of contents, consignment status, or other pertinent information as deemed necessary to properly track the Equipment.

13. The system of claim 1 in which said computer is programmed to display filterable dashboards with multiple options to view or manipulate data.

14. A check-in and check-out station for use in a system for tracking medical equipment into and out of a health care facility comprising:
a user interface;
a camera for taking photos of equipment;
a computer programmed for entering data concerning the equipment into the computer, accepting said photos of the equipment and linking said photos to said data, and preparing identifying labels for said equipment based on said data;
a viewable display;
and a printer operably connected to said computer to facilitate printing said labels for attachment to said equipment or a package containing said equipment.

15. The station of claim 10 which also includes a code scanner for scanning code placed on a label on equipment.

16. A method for tracking medical equipment into and out of a health care facility comprising:
Providing a check-in & check-out station for medical equipment comprising:
a user interface,
a camera for taking at least one photo of the equipment,
a computer programmed for entering data concerning the equipment into the computer, accepting said photos of the equipment and linking said photos to said data, and preparing identifying labels for said equipment based on said data,
a viewable display,
and a printer operably connected to said computer to facilitate printing said labels for attachment to said equipment or a package containing said equipment;
linking a server for storing said data and said linked photos, to said check-in & check-out station computer;
said server and said computer being programmed to provide authorized medical equipment vendors and medical facility users, access to said data and photos from any internet enabled location and from said check-in & check-out station;
receiving into said server data and photos generated by users checking equipment into said facility using said check-in & check-out station;
making said data and photos available to authorized medical equipment vendors and medical facility users.

17. The method of claim 16 which includes applying to said equipment labels generated by said computer.

18. The method of claim 17 in which said computer is programmed to generate a computer recognizable code which is included with said data, and also to print said code on said labels, whereby said code can be scanned by an authorized user or vendor to determine the contents of a package containing said equipment.

19. The method of claim 18 in which said check-in and check-out station includes a scanner for scanning and recognizing said code.

20. Computer software for tracking medical equipment into and out of a health care facility comprising:
a user interface module, facilitating use of said software by a user checking medical equipment into and out of a health care facility;
a camera linking module for linking said software to a camera used for taking at least one photo of said medical equipment;
a data storage module;
a data assembly module for receiving photographs taken of said medical equipment and for receiving data entered identifying said medical equipment being checked into or out of a health care facility;
a labeling engine linked to and activated by said data receiving module for preparing identifying labels for said medical equipment based on said data in said data assembly module;
a data addressing module for addressing and transferring said data, said photographs and said label to an identifying address in said storage module;
a display driver facilitates displaying data;
a printer control module to facilitate printing said labels for attachment to said medical equipment or a package containing said medical equipment;
a user authorization module for receiving identification information for authorized medical equipment vendors and medical facility users and allowing said users access to said data and photos from any internet enabled location.

21. Computer software of claim 20 which includes a code generator, linked to said labeling engine, which generates a computer recognizable code which is included with said data, and also to print said code on said label, whereby said code can be scanned by an authorized user or vendor to determine the contents of a package containing said equipment.

22. Computer software of claim 21 which includes a scanner control module for scanning and recognizing said code.

23. Computer software of claim 22 which includes a notification module to provide and allow users to select the option of receiving notifications of particular events related to equipment which is entered into the system.

24. Computer software of claim 23 which includes which also provides an “auto-fill” capability, whereby information pertinent to particular equipment or surgical cases which has been previously entered into the system auto-fills that information for the user as equipment is checked in.

25. Computer software of claim 24 which includes said user interface module prompts the user for entry of additional data information selected from the group consisting of the surgeon name, field of practice, surgery date, surgery time, procedure performed, operative side, patient initials, user representative name, user company name, loaner fee, tray weight, tray dimensions, directions for use, instructions for processing, instructions for handling, list of contents, consignment status, or other pertinent information as deemed necessary to properly track the Equipment.

26. Computer software of claim 25 which includes a dashboard display module to display filterable dashboards with multiple options to view or manipulate data.

27. Computer software of claim 26 which includes said user interface module programmed to receive entry of anticipated check out dates for equipment, and to automatically notify the relevant users.

28. Computer software of claim 20 which includes said user interface module programmed to receive entry of anticipated check out dates for equipment, and to automatically notify the relevant users.
